(19) United States
(12) Patent Application Publication
(10) Pub. No.: US 2025/0332448 A1
(43) Pub. Date: Oct. 30, 2025

(54) RADIOTHERAPY SYSTEM AND METHOD FOR CONTROLLING SAME

(71) Applicant: Hitachi High-Tech Corporation, Tokyo (JP)

(72) Inventors: Hidetaka SUGIYAMA, Tokyo (JP); Takao KIDANI, Tokyo (JP); Shin YANAGISAWA, Tokyo (JP)

(21) Appl. No.: 18/855,363

(22) PCT Filed: Feb. 16, 2023
(86) PCT No.: PCT/JP2023/005540

§ 371 Date: Oct. 9, 2024

(30) Foreign Application Priority Data
Jun. 15, 2022 (JP) 2022-096781

(51) Int. Cl. A61N 5/10

(52) U.S. Cl. CPC A61N 5/1049; A61N 5/1081; A61N 2005/1074

(57) ABSTRACT

A radiotherapy system including a gantry that includes a beam delivery device and imaging devices to includes: a parameter selection unit that selects an imaging parameter including an imaging start angle that is a gantry angle when the imaging device starts imaging, an imaging end angle that is a gantry angle when the imaging device ends imaging, and a rotation direction of the gantry; and a control unit that controls the imaging device by using a predetermined imaging parameter selected by the parameter selection unit, in which the parameter selection unit generates one or more candidates of the imaging parameter and selects, as the predetermined imaging parameter, a candidate that reduces a total movement amount of the gantry required for imaging by the imaging device and therapeutic beam delivery by the beam delivery device among the generated candidates of the imaging parameter.

### TECHNICAL FIELD

The present invention relates to a radiotherapy system and a method for controlling the same.

### BACKGROUND ART

Image-guided radiotherapy (IGRT) is a treatment in which an image at the time of treatment planning and an image captured immediately before or during treatment are collated, and misalignment of a detected position is automatically corrected and therapeutic beam delivery is performed. Since a situation of a target volume when a treatment plan is created and a situation of the target volume immediately before the treatment change, the target volume is imaged immediately before the treatment and a deviation from the treatment plan is detected.

PTL 1 discloses a technology of predicting a time required for radiotherapy based on a rotation angle of a gantry and a beam delivery angle of a beam delivery gate and displaying the predicted time.

### CITATION LIST

### Patent Literature

PTL 1: JP 6887889 B2

### SUMMARY OF INVENTION

### Technical Problem

After an imaging field for determining a position of the target volume immediately before the treatment, a treatment field for delivering a predetermined amount of beam to the target volume according to the treatment plan is implemented. In order to efficiently advance the radiotherapy, it is preferable to rotate the gantry to the beam delivery angle with as little movement amount as possible after the imaging field and to quickly move to the treatment field. For this purpose, it is necessary for a user such as a doctor or a radiologist to study and set an appropriate imaging parameter. Therefore, a burden on the user for setting an appropriate imaging parameter is large. Furthermore, the longer the time required to set an appropriate imaging parameter, the longer the treatment time, and thus the greater the burden on a patient receiving the radiotherapy.

The present invention has been made in view of the above problems, and an object of the present invention is to provide a radiotherapy system and a method for controlling the same capable of efficiently selecting an appropriate imaging parameter.

### Solution to Problem

In order to solve the above problems, a radiotherapy system according to the present invention is a radiotherapy system including a gantry that includes a beam delivery device and an imaging device, the radiotherapy system including: a parameter selection unit that selects an imaging parameter including an imaging start angle which is a gantry angle when the imaging device starts imaging, an imaging end angle which is a gantry angle when the imaging device ends imaging, and a rotation direction of the gantry; and a control unit that controls the imaging device by using a predetermined imaging parameter selected by the parameter selection unit, in which the parameter selection unit generates one or more candidates of the imaging parameter and selects, as the predetermined imaging parameter, a candidate that reduces a total movement amount of the gantry required for imaging by the imaging device and therapeutic beam delivery by the beam delivery device among the generated candidates of the imaging parameter.

### Advantageous Effects of Invention

According to the present invention, it is possible to select a predetermined imaging parameter with which the total movement amount of the gantry becomes smaller.

### BRIEF DESCRIPTION OF DRAWINGS

FIG. 1 is an overall configuration diagram of a radiotherapy system.

FIG. 2 is an explanatory view illustrating an internal configuration of a gantry.

FIG. 3 is a plan view of the gantry.

FIG. 4 is an explanatory view illustrating a state in which rotation of the gantry is restricted.

FIG. 5 is an explanatory view in a case where X-ray imaging is performed along two axes.

FIG. 6 is an explanatory view in a case where X-ray imaging is performed along one axis.

FIG. 7 illustrates an example of a table for managing an imaging range of cone-beam computed tomography (CBCT).

FIG. 8 is a functional configuration diagram of a control system of the radiotherapy system.

FIG. 9 is a flowchart of radiotherapy processing.

FIG. 10 is a flowchart of processing of setting an imaging parameter.

FIG. 11 illustrates an example of a table for managing candidates of the imaging parameter.

FIG. 12 is an explanatory view schematically illustrating a relationship among a current angle of the gantry, an imaging start angle, and an imaging end angle.

FIG. 13 is an explanatory diagram illustrating an example of an order in a case where a therapeutic beam is delivered from a plurality of beam delivery angles according to a second embodiment.

FIG. 14 illustrates an example of a table for managing candidates of the imaging parameter for each case.

FIG. 15 is a flowchart of processing of calculating the imaging parameter for each beam delivery angle and determining a beam delivery order.

FIG. 16 is an explanatory diagram illustrating a setup angle of radiology (RAD) imaging according to a third embodiment.

FIG. 17 is a flowchart of processing of setting an imaging parameter.

FIG. 18 is an explanatory diagram illustrating an example of an order in a case where a therapeutic beam is delivered from a plurality of beam delivery angles according to a fourth embodiment.

FIG. 19 is a flowchart of processing of setting an imaging parameter.

FIG. 20 is an explanatory view in a case where a rotation restriction angle TP is not set for a gantry according to a fifth embodiment.

### DESCRIPTION OF EMBODIMENTS

Hereinafter, embodiments of the present invention will be described with reference to the drawings. A radiotherapy system 1000 according to the present embodiment automatically determines imaging parameters (including an imaging start gantry angle, an imaging end gantry angle, and a gantry rotation direction) with which a rotational movement amount of a gantry 11 becomes smaller in consideration of a beam delivery start angle of a therapeutic beam, and provides the determined imaging parameters to a user such as a doctor or a radiologist.

In the present embodiment, a particle beam will be described as an example of the therapeutic beam. Examples of the particle beam include a neutron beam, a proton (hydrogen) beam, a helium beam, and a carbon beam. However, the present embodiment can be applied not only to a particle beam but also to an X-ray or an electron beam. Hereinafter, the therapeutic beam may be referred to as a particle beam.

In the radiotherapy system according to an aspect of the present disclosure, the parameter selection unit may be configured to generate one or more candidates of the imaging parameter in which a difference between a beam delivery start angle and the imaging end angle is within a predetermined range, the beam delivery start angle being a gantry angle at which therapeutic beam delivery is performed by the beam delivery device, and select, as the predetermined imaging parameter, a candidate that reduces the total movement amount of the gantry required for imaging by the imaging device and the therapeutic beam delivery by the beam delivery device among the generated candidates of the imaging parameter.

In the radiotherapy system according to an aspect of the present disclosure, the parameter selection unit may be configured to select an imaging parameter that reduces a total movement amount from a current angle of the gantry to the beam delivery start angle among the generated candidates of the imaging parameter.

In the radiotherapy system according to an aspect of the present disclosure, the parameter selection unit may be configured to select, as the predetermined imaging parameter, a candidate of the imaging parameter that reduces a total movement amount of the gantry from a current angle of the gantry until transitioning to the therapeutic beam delivery by the beam delivery device through the imaging by the X-ray imaging device, among the generated candidates of the imaging parameter.

The radiotherapy system according to an aspect of the present disclosure may include a rotating gantry for which a rotation restriction angle that restricts rotation beyond one full rotation is set, and the parameter selection unit may be configured to generate at least one candidate of the imaging parameter in which the rotation restriction angle is not included in a range from the imaging start angle to the imaging end angle.

The radiotherapy system according to an aspect of the present disclosure may include a rotating gantry for which a rotation restriction angle that restricts rotation beyond one full rotation is set, and the parameter selection unit may be configured to generate, in a case where the rotation restriction angle is within a range from the imaging start angle to the imaging end angle among the generated imaging parameters, other candidates of the imaging parameter in which the rotation restriction angle is not included in the range from the imaging start angle to the imaging end angle, and select, as the predetermined imaging parameter, a candidate that reduces a total movement amount of the gantry required for the imaging by the imaging device and the therapeutic beam delivery by the beam delivery device among the generated other candidates of the imaging parameter.

In the radiotherapy system according to an aspect of the present disclosure, the parameter selection unit may be configured to generate the other candidates of the imaging parameter such that the imaging start angle is the rotation restriction angle.

In the radiotherapy system according to one aspect of the present disclosure, the imaging device may include a plurality of X-ray generation units provided so as to sandwich a beam delivery unit, and a plurality of X-ray imaging units provided in the gantry at positions facing the X-ray generation units, and imaging may be performed by at least one set of the X-ray generation unit and the X-ray imaging unit among a plurality of sets of the X-ray generation units and the X-ray imaging units disposed to face each other.

In the radiotherapy system according to an aspect of the present disclosure, the parameter selection unit may be configured to generate a candidate of the imaging parameter in a case where imaging is performed by any one set of the X-ray generation unit and the X-ray imaging unit among the plurality of sets of the X-ray generation units and the X-ray imaging units disposed to face each other, and a candidate of the imaging parameter in a case where imaging is performed by all the sets of the X-ray generation units and the X-ray imaging units among the plurality of sets of the X-ray generation units and the X-ray imaging units disposed to face each other.

In the radiotherapy system according to an aspect of the present disclosure, the imaging device may be configured to select any one of a two-dimensional image acquisition mode in which a two-dimensional image including a beam delivery target is acquired by an X-ray and a three-dimensional image acquisition mode in which a three-dimensional image including the beam delivery target is acquired by an X-ray.

In the radiotherapy system according to an aspect of the present disclosure, the parameter selection unit may be configured to generate, in a case where the two-dimensional image acquisition mode is selected, a candidate of the imaging parameter that reduces a movement amount of the gantry to a beam delivery start angle among a plurality of imaging angles prepared in advance, the beam delivery start angle being a gantry angle at which the therapeutic beam delivery is performed by the beam delivery device.

In the radiotherapy system according to an aspect of the present disclosure, a case where the difference between the beam delivery start angle, which is a gantry angle at which the therapeutic beam delivery is performed by the beam delivery device, and the imaging end angle is within the predetermined range may include a case where the beam delivery start angle and the imaging end angle match each other.

In the radiotherapy system according to an aspect of the present disclosure, the control unit may cause the beam delivery device to deliver the therapeutic beam to a beam delivery target based on a misalignment amount between a current image of the beam delivery target captured by the imaging device and a reference image at the time of treatment planning.

A method for controlling a radiotherapy system according to an aspect of the present disclosure is a method for controlling a radiotherapy system including a gantry that includes a beam delivery device and an imaging device, the method including: generating candidates of an imaging parameter including an imaging start angle which is a gantry angle when the imaging device starts imaging, an imaging end angle which is a gantry angle when the imaging device ends imaging, and a rotation direction of the gantry; and selecting, as a predetermined imaging parameter, a candidate that reduces a total movement amount of the gantry required for imaging by the imaging device and therapeutic beam delivery by the beam delivery device among the generated candidates of the imaging parameter.

### First Embodiment

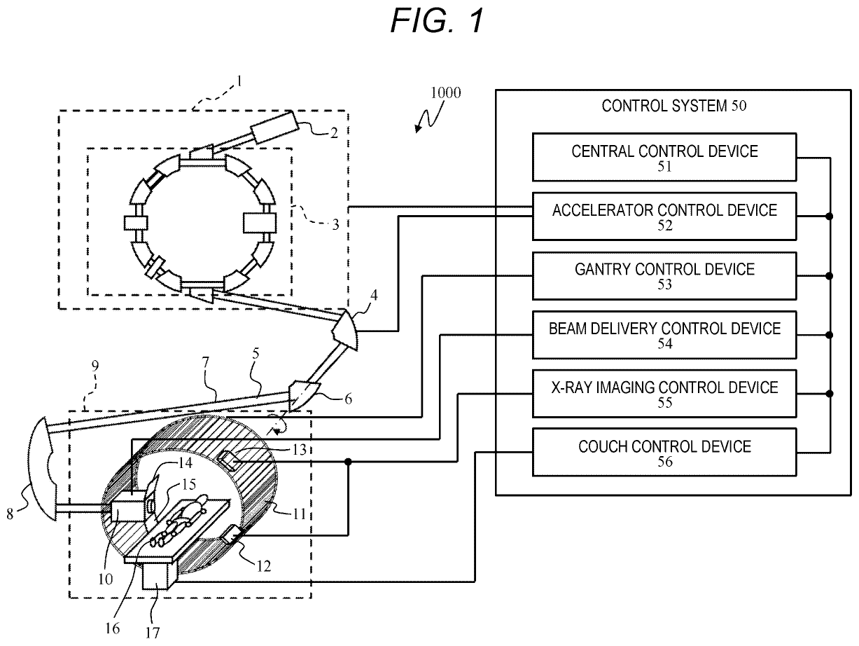

A first embodiment will be described with reference to FIGS. 1 to 12. FIG. 1 illustrates an overall configuration of a radiotherapy system. The radiotherapy system mainly includes a therapeutic beam generator 1, a particle beam transport system 5, and a treatment room 9.

The therapeutic beam generator 1 includes, for example, an ion source (not illustrated), a linac 2 as a pre-acceleration device, and a circular acceleration device (for example, a synchrotron) 3. In the present embodiment, the synchrotron 3 is described as an example of the circular acceleration device, but another circular accelerator such as a cyclotron or a synchronous cyclotron may be used. The particle beam transport system 5 includes, for example, a particle beam path 7 connecting the therapeutic beam generator 1 and the treatment room 9, a quadrupole magnet (not illustrated) provided in the middle of the particle beam path 7, and bending magnets 4, 6, and 8.

The treatment room 9 includes, for example, a substantially tubular gantry 11, a beam delivery device 10, X-ray generators 12 and 13, X-ray detectors 14 and 15, and a couch 17.

A therapeutic beam (proton beam) generated from the ion source is pre-accelerated by the linac 2 and is injected to the synchrotron 3. The therapeutic beam further accelerated by the synchrotron 3 is extracted to the particle beam transport system 5.

The therapeutic beam extracted from the synchrotron 3 is focused by the quadrupole magnet (not illustrated) while passing through the particle beam path 7, and changed in direction by the bending magnets 4, 6, and 8 to be injected to the treatment room 9. A part of the particle beam transport system 5 (the bending magnets 6 and 8 and a part of the particle beam path 7) is installed in the gantry 11 so as to rotate integrally with the gantry 11.

The therapeutic beam injected to the treatment room 9 from the particle beam transport system 5 is extracted from the beam delivery device 10 toward a beam delivery target 16 disposed on the couch 17. The beam delivery device 10 is installed in the gantry 11, and can be adjusted to an arbitrary angle by rotation control of the gantry 11.

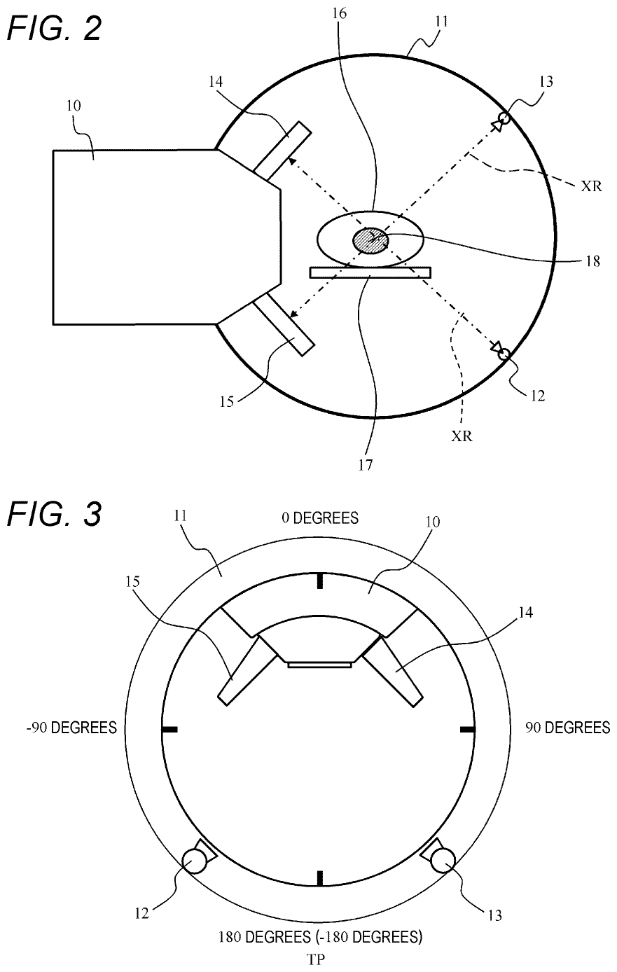

An internal configuration of the gantry 11 will be described with reference to FIG. 2. The beam delivery device 10 that extracts the therapeutic beam toward a target 18 in the beam delivery target 16 is installed in the gantry 11. Further, a plurality of sets of X-ray imaging devices are arranged so as to sandwich a particle beam delivery port of the beam delivery device 10. That is, the X-ray imaging device including a combination of the X-ray generators 12 and 13 and the X-ray detectors 14 and 15 is disposed in the gantry 11 such that X-ray paths are orthogonal to each other. In the illustrated example, the X-ray generator 12 and the X-ray detector 14 facing each other form one set, and the X-ray generator 13 and the X-ray detector 15 facing each other form another set.

In order to deliver the therapeutic beam to the target 18, the beam delivery target 16 is accurately disposed at a preset position. A user such as a doctor or a radiologist moves the couch 17 while confirming X-ray images of the beam delivery target 16 obtained by the X-ray imaging devices (12 and 14) and (13 and 15), and disposes the beam delivery target 16 at a position set in advance in a treatment plan.

The beam delivery device 10 extracts the therapeutic beam toward the beam delivery target 16 to form dose distribution suitable for a shape of the target 18 in the beam delivery target 16.

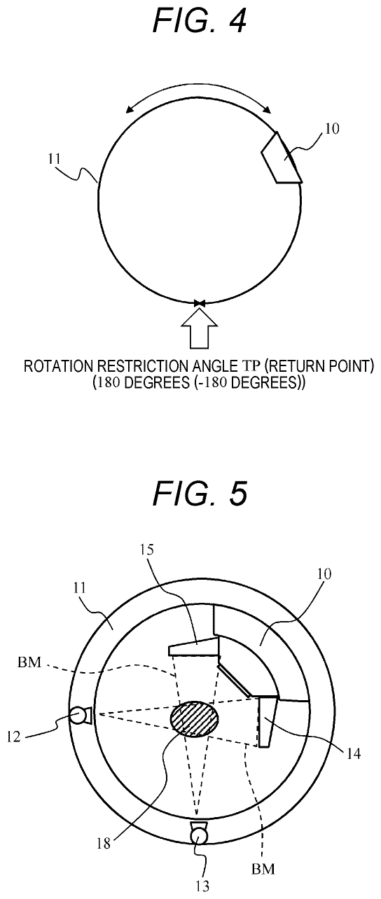

FIG. 3 is a plan view of the gantry 11. The upper side of the drawing is defined as a gantry angle of 0 degrees. The gantry 11 may rotate clockwise (CW) by 90 degrees, up to 180 degrees. Herein, angles from 0 degrees illustrated on the upper side of FIG. 3 in a clockwise direction are described, and angles from 0 degrees in a counterclockwise direction are described in parentheses following the angles. The angles in the counterclockwise direction are given a minus sign. One full clockwise rotation from a reference angle of 0 degrees results in 360 degrees. The gantry 11 can rotate counterclockwise (CCW) by −90 degrees (270 degrees when rotating clockwise), up to −180 degrees (180 degrees when rotating clockwise). In the present embodiment, a configuration in which the gantry 11 cannot rotate more than one rotation in both the clockwise direction and the counterclockwise direction will be described as an example. As also illustrated in FIG. 4, the gantry 11 according to the present embodiment is configured to turn back at a rotation restriction angle TP. The rotation of the gantry 11 is restricted by a restriction on a device structure. As described below, in a case where a rotation start angle of the gantry 11 is set to the rotation restriction angle TP, the gantry 11 can rotate by less than 360 degrees (for example, up to 359 degrees).

FIG. 5 is an explanatory view in a case where X-ray imaging is performed along two axes. The X-ray imaging along two axes is to acquire an image including the target 18 of the beam delivery target 16 by using two sets of X-ray imaging devices (12 and 14) and (13 and 15).

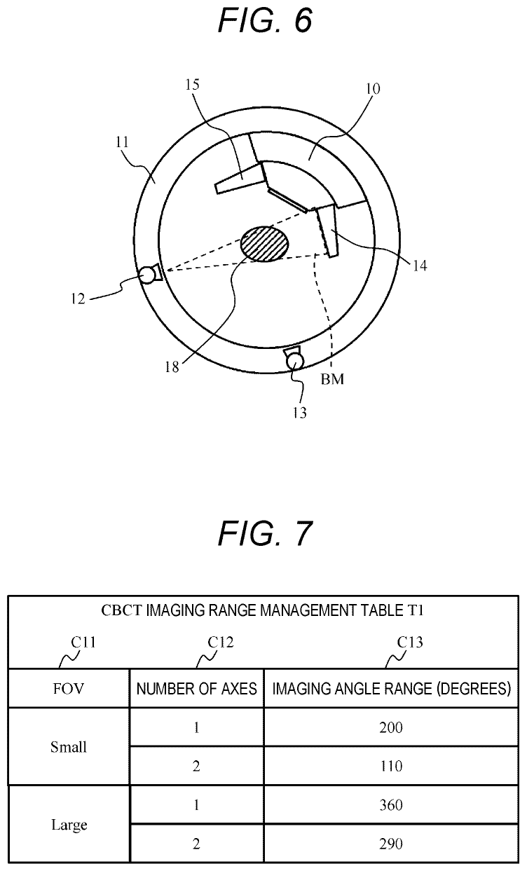

FIG. 6 is an explanatory view in a case where X-ray imaging is performed along one axis. The X-ray imaging along one axis is to acquire an image including the target 18 of the beam delivery target 16 by using any one of two sets of X-ray imaging devices (12 and 14) and (13 and 15).

As described below, in the case of computed tomography (CT) in which imaging is performed with a cone beam (hereinafter, referred to as cone-beam computed tomography (CBCT)), an angle range required for the X-ray imaging along two axes can be made smaller than an angle range required for the X-ray imaging along one axis. Therefore, the X-ray imaging along two axes is generally performed. However, there is a possibility that the X-ray imaging along one axis is performed using any one of the two sets of X-ray imaging devices (12 and 14) and (13 and 15). Therefore, the radiotherapy system 1000 according to the present embodiment can cope with both the X-ray imaging along one axis and the X-ray imaging along two axes.

FIG. 7 illustrates an example of a table T1 for managing an imaging range of the CBCT. The CBCT imaging range management table T1 manages, for example, FOV_C11, the number of axes C12, and an imaging angle range C13 in association with each other.

FOV_C11 is an imaging field of view. In the present embodiment, for example, two types are prepared: a case where the imaging field of view is small (Small) and a case where the imaging field of view is large (Large). Three or more FOVs may be available.

The number of axes C12 indicates how many X-ray imaging devices are used out of the two sets of X-ray imaging devices (12 and 14) and (13 and 15). As described above, the number of axes C12 includes “one axis” and “two axes”.

The imaging angle range C13 is an angle range required for imaging a necessary range. For example, in the case of the X-ray imaging along one axis with the FOV of “Small”, it is necessary to rotate the gantry 11 by 200 degrees to perform imaging. On the other hand, in the case of the X-ray imaging along two axes with the FOV of “Small”, it is possible to obtain an image of a necessary range only by rotating the gantry 11 by 110 degrees.

In a case where the FOV is “Large”, it is necessary to rotate the gantry 11 by 360 degrees in the X-ray imaging along one axis. On the other hand, in a case where the FOV is “Large”, the gantry 11 may be rotated by 290 degrees in the X-ray imaging along two axes.

As described above, in a case where the FOVs are the same, a gantry rotation angle required for imaging is smaller in the X-ray imaging along two axes than in the X-ray imaging along one axis. Furthermore, the larger the FOV, the larger the gantry rotation angle required for imaging.

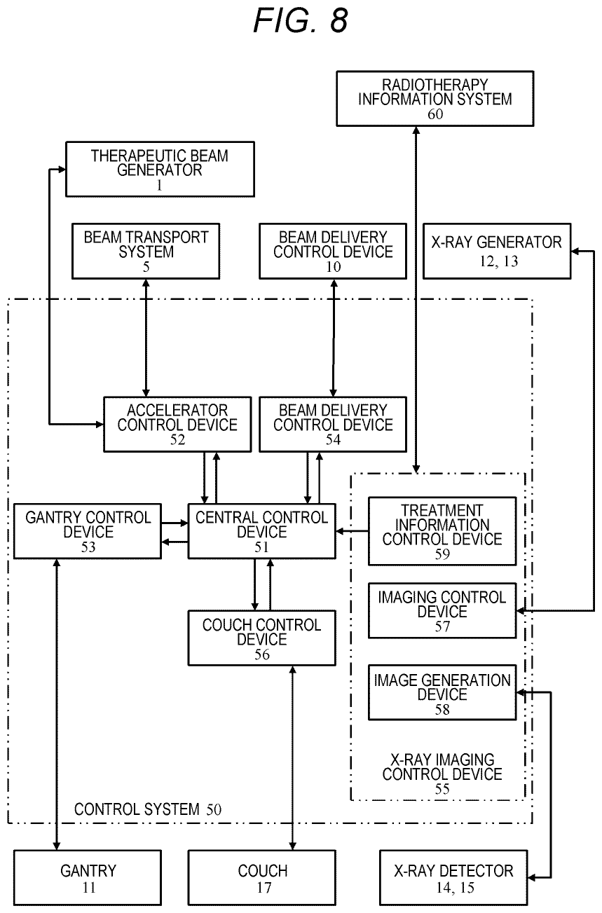

A configuration of a control block of a control system 50 will be described with reference to FIG. 8. The control system 50 includes, for example, a central control device 51, an accelerator control device 52, a gantry control device 53, a beam delivery control device 54, an X-ray imaging control device 55, and a couch control device 56, and is communicably connected to a radiotherapy information system 60. A beam used for imaging a target volume is referred to as an imaging beam. In the present embodiment, an X-ray is taken as an example of an imaging delivered beam.

The central control device 51 is connected to the accelerator control device 52, the gantry control device 53, the beam delivery control device 54, the X-ray imaging control device 55, and the couch control device 56, and controls the control system 50 by transmitting and receiving necessary information therebetween.

The accelerator control device 52 is connected to and controls the therapeutic beam generator 1 and the particle beam transport system 5. The gantry control device 53 is connected to the gantry 11 and controls the rotation of the gantry 11. The beam delivery control device 54 controls equipment in the beam delivery device 10 to deliver the therapeutic beam to the target 18 of the beam delivery target 16 from the beam delivery device 10 such that predetermined dose distribution is formed in the beam delivery target 16.

The X-ray imaging control device 55 is connected to each of the above-described two sets of X-ray imaging devices (12 and 14) and (13 and 15), and controls the two sets of X-ray imaging devices (12 and 14) and (13 and 15). The couch control device 56 is connected to the couch 17 and controls the couch 17.

The X-ray imaging control device 55 includes an imaging control device 57, an image generation device 58, and a treatment information control device 59. The imaging control device 57 controls operations of the X-ray generators 12 and 13.

Each of the X-ray generators 12 and 13 that have received an X-ray imaging signal delivers an X-ray, which is an example of the imaging beam, to the target 18. The X-rays transmitted through the target 18 are detected by the facing X-ray detectors 14 and 15. The X-ray detectors 14 and 15 convert the detected X-rays into electric signals and transmit the electric signals to the image generation device 58. The image generation device 58 generates an X-ray captured image based on the pieces of electric signal information obtained from the X-ray detectors 14 and 15, and displays the generated image data.

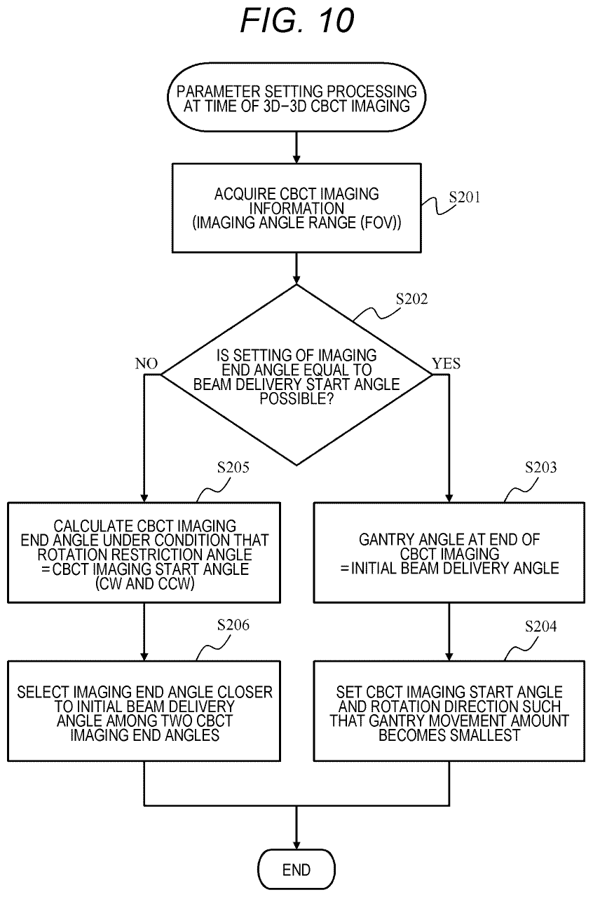

The treatment information control device 59 serving as a “parameter selection unit” acquires the treatment plan from the radiotherapy information system 60, selects an imaging parameter, and inputs the imaging parameter to the central control device 51. Processing described below with reference to FIGS. 10, 15, 17, and 19 can also be understood as an example of a “parameter control unit”.

When a beam delivery permission signal transitions to an on state, the central control device 51 serving as a “control unit” transmits a beam delivery start signal to the accelerator control device 52 and the beam delivery control device 54. The accelerator control device 52 and the beam delivery control device 54 respectively control the therapeutic beam generator 1 and the beam delivery device 10 to deliver the therapeutic beam to the target 18.

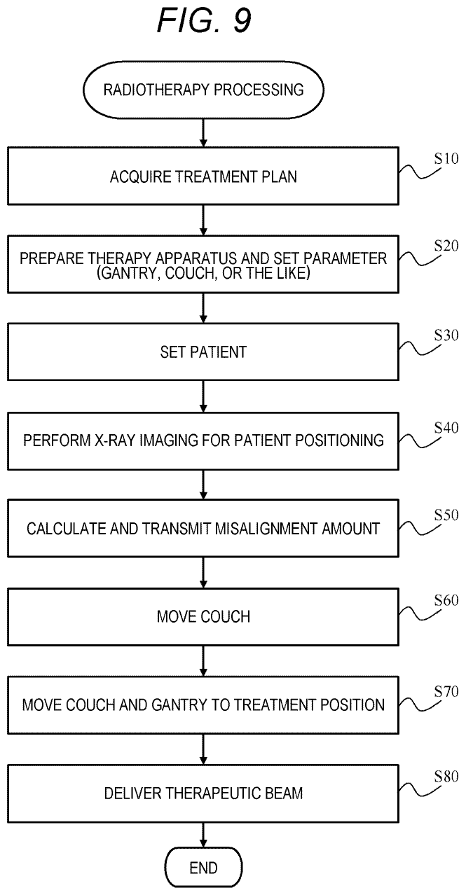

Radiotherapy processing will be described with reference to a flowchart of FIG. 9. The control system 50 acquires the treatment plan from the radiotherapy information system 60 (S10), prepares a therapy apparatus, and sets the imaging parameter (S20). In step S20, a position (angle) of the gantry 11 and a position and posture of the couch 17 are controlled. A method for setting the imaging parameter in step S20 is described below with reference to FIG. 10.

A patient as the beam delivery target 16 is fixed on the couch 17 in a predetermined posture (S30). This series of operations is referred to as a patient setup. Step S30 is not processing executed by the control system 50 but is manually executed by the user and an assistant. However, a setup assist robot or the like can also be used to fix the patient to the couch 17, in which case the control system 50 can control the setup assist robot.

The control system 50 acquires an X-ray image (an example of a current image) for patient positioning (S40). The X-ray image for patient positioning is an X-ray image indicating the current position of the target 18 imaged by the X-ray imaging device. The control system 50 acquires an X-ray image (an example of a reference image) of the patient acquired at the time of treatment planning from a treatment planning device. Then, the control system 50 calculates a misalignment amount between the position of the target 18 in the treatment plan and the latest position of the target 18 acquired in step S40 based on a misalignment amount between the reference image at the time of the treatment planning and the current image captured by the X-ray imaging device, and outputs the calculated misalignment amount to the couch control device 56 (S50). When the misalignment amount is received from the control system 50, the couch control device 56 moves the couch 17 to eliminate the misalignment amount (S60).

The control system 50 moves the couch 17 and the gantry 11 to a treatment position (S70). In step S70, in the case of radiology (RAD) imaging, the gantry 11 is moved from a setup angle to a beam delivery angle (S70). When the treatment is ready to be started, the control system 50 causes the beam delivery device 10 to deliver the therapeutic beam to the beam delivery target 16 (S80).

The flowchart of FIG. 10 illustrates processing of setting the imaging parameter. This processing is executed as a part of step S20 described in FIG. 9. In this processing, a parameter of CBCT imaging in a case where radiotherapy is performed by a 3D-3D method is set. Here, 3D-3D refers to processing of acquiring a three-dimensional image including the target 18 by using the CBCT at the time of treatment planning and immediately before the treatment.

The control system 50 acquires a value of the FOV (Small or Large) as basic information of the CBCT imaging (S201). The control system 50 determines whether or not the CBCT imaging is possible in a case where an imaging end angle is set to a beam delivery start angle (S202).

If the imaging end angle matches the beam delivery start angle, transition from an imaging field to a treatment field can be made in a short time, so that a time required for the radiotherapy can be shortened. On the other hand, the restriction angle TP is set for the rotation of the gantry 11, and thus, the gantry 11 cannot rotate beyond the restriction angle TP. In a case where the imaging end angle matches the beam delivery start angle, an imaging start angle is naturally determined from the value of the FOV and the number of axes. In a case where the restriction angle TP is included in a range from the imaging start angle to the imaging end angle determined based on the value of the FOV and the number of axes, the gantry 11 cannot move on the path.

The imaging end angle and the beam delivery start angle may strictly or substantially match each other. The imaging end angle and the beam delivery start angle substantially matching each other means, for example, the imaging end angle and the beam delivery start angle matching each other with an error within an allowable range defined in Digital Imaging and Communications in Medicine (DICOM). In addition to a case where the imaging end angle and the beam delivery start angle are substantially the same as each other, a case where a difference between the beam delivery start angle and the imaging end angle is within a predetermined range is also included in the scope of the present embodiment. For example, a case where the imaging end angle is different from the beam delivery start angle by several degrees to several tens of degrees is also included in the scope of the present embodiment. Also in this case, the transition from the imaging field to the treatment field can be made in a relatively short time.

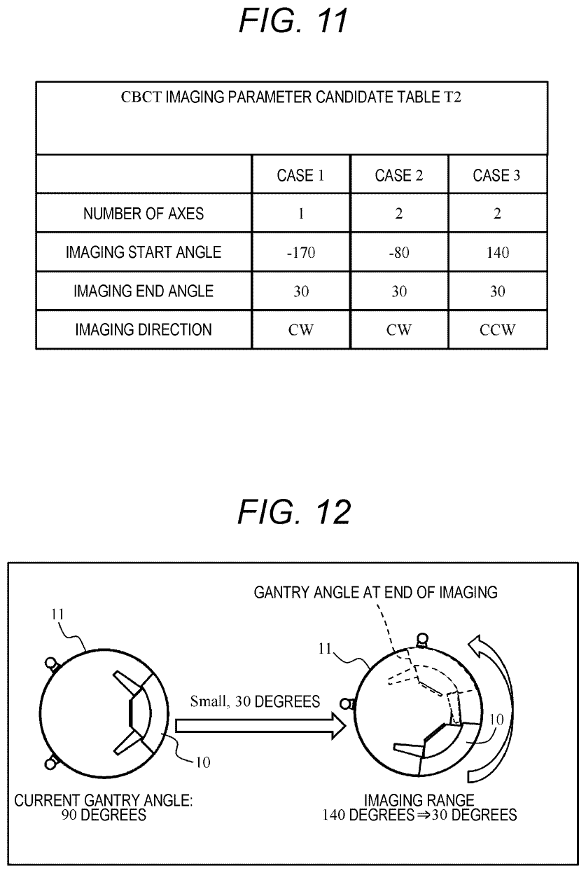

In a case where it is determined in step S202 that the CBCT imaging is possible in a case where the imaging end angle is set to the beam delivery start angle (S202: YES), the control system 50 sets the imaging end angle of the CBCT to be an angle (beam delivery angle) at which the first particle beam (therapeutic beam) is delivered (S203). Then, the control system 50 sets the imaging start angle of the CBCT and a rotation direction of the gantry 11 such that a movement amount (rotation amount) of the gantry 11 becomes smaller (S204). An example of the setting method is described below with reference to FIG. 11.

On the other hand, in a case where the CBCT imaging cannot be performed when the imaging end angle matches the beam delivery start angle (S202: NO), the control system 50 calculates the imaging end angle in the clockwise direction and the imaging end angle in the counterclockwise direction when the imaging start angle of the CBCT matches the rotation restriction angle TP (S205). The control system 50 selects an imaging end angle closer to the beam delivery angle of the first particle beam determined in the treatment plan out of the imaging end angle in the clockwise direction and the imaging end angle in the counterclockwise direction (S206).

FIG. 11 illustrates an example of a table T2 for managing candidates of the imaging parameter. The candidate table T2 is applied in a case where an angle at which beam delivery is to be performed is initially determined or in a case where there is only one beam delivery angle. Hereinafter, a case where the beam delivery angle determined in the treatment plan is 30 degrees, the FOV is “Small”, and the current gantry angle is 90 degrees will be described. The current gantry angle is an initial value of the gantry angle before the imaging parameter is selected.

Here, it is assumed that the imaging end angle and the beam delivery start angle match each other in order to shorten a time required for the treatment. In a case where the FOV is “Small”, as described in FIG. 7, the imaging angle range along one axis is 200 degrees, and the imaging angle range along two axes is 110 degrees.

In Case 1 where the CBCT imaging is performed along one axis, imaging is performed by rotating the gantry 11 clockwise by 200 degrees to the imaging end angle of 30 degrees. Therefore, the imaging start angle is 190 degrees (−170 degrees). When the CBCT imaging is performed along two axes, there are two cases of clockwise rotation and counterclockwise rotation.

In Case 2 where the CBCT imaging is performed by rotating the gantry 11 clockwise to the imaging end angle of 30 degrees, the imaging start angle is 280 degrees (−80 degrees) (see FIG. 3). In Case 3 where the CBCT imaging is performed by rotating the gantry 11 counterclockwise to the imaging end angle of 30 degrees, the imaging start angle is 140 degrees.

As a given value, the current gantry angle is 90 degrees. When the current gantry angle of 90 degrees is compared with the imaging start angle of 280 degrees (−80 degrees) in Case 2 and the imaging start angle of 140 degrees in Case 3, 140 degrees is closer. Therefore, as illustrated in FIG. 12, the imaging parameter defined in Case 3 is selected. When the imaging parameter of Case 3 is set, the gantry 11 rotates clockwise by 50 degrees from the current angle of 90 degrees to reach the imaging start angle of 140 degrees. Then, the CBCT imaging is started. The CBCT imaging is continued while the gantry 11 rotates by 110 degrees counterclockwise from the imaging start angle of 140 degrees, and the CBCT imaging ends when the gantry 11 reaches the imaging end angle of 30 degrees. Therefore, before the start of imaging, the gantry 11 rotates by 50 degrees from 90 degrees to 140 degrees, and rotates by 110 degrees from 140 degrees to 30 degrees during imaging. The total movement amount of the gantry 11 is 160 (=50+110) degrees. In Case 2, the gantry 11 rotates by 170 degrees counterclockwise from the current angle of 90 degrees to 280 degrees (−80 degrees) to reach the imaging start angle, and rotates by 110 degrees clockwise therefrom to perform the CBCT imaging. Therefore, the total movement amount of the gantry 11 is 280 (170+110) degrees. Since the total movement amount of Case 3 is smaller than the total movement amount of Case 2, wear and the like caused by the movement of the gantry 11 can be reduced, and power consumption can also be reduced.

Normally, the CBCT imaging along two axes is selected in order to shorten a treatment time, but in a case where any one of the X-ray imaging devices is out of order, the CBCT imaging along one axis is performed. In this case, the imaging parameter of Case 1 is selected.

According to the present embodiment configured as described above, since it is possible to automatically select and set the imaging parameter that shortens the time required for the CBCT imaging, usability for the user is improved. Furthermore, according to the present embodiment, since the time required for the CBCT imaging can be shortened, the time required for treatment can also be shortened, so that a burden on the patient can be reduced. Furthermore, in the present embodiment, since the total movement amount when the gantry 11 moves from the imaging field to the treatment field can be reduced, wear and the like caused by the movement of the gantry 11 can be reduced, so that maintenance frequency can be reduced. Furthermore, since the total movement amount of the gantry 11 is reduced, the power consumption can also be reduced. An operation cost of the radiotherapy system 1000 can be reduced by the reduction in maintenance frequency and the reduction in power consumption.

In the present embodiment, it has been described that a candidate of the imaging parameter that reduces the total movement amount of the gantry 11 (the total movement amount when the gantry 11 moves from the imaging field to the treatment field) is selected from the candidates of the imaging parameter. The candidates of the imaging parameter that reduce the total movement amount include a candidate that reduces the total movement amount the most. The scope of the present disclosure includes not only the candidate of the imaging parameter that reduces the total movement amount the most but also a candidate of the imaging parameter that reduces the total movement amount more than before although the candidate does not reduce the total movement amount the most.

In the present embodiment, the gantry that cannot rotate beyond one full rotation due to a mechanical restriction of a movable range of the gantry 11 has been described as an example. However, the present embodiment can be applied even when the gantry 11 is configured to be rotatable by 360 degrees or more as in other embodiments described below.

### Second Embodiment

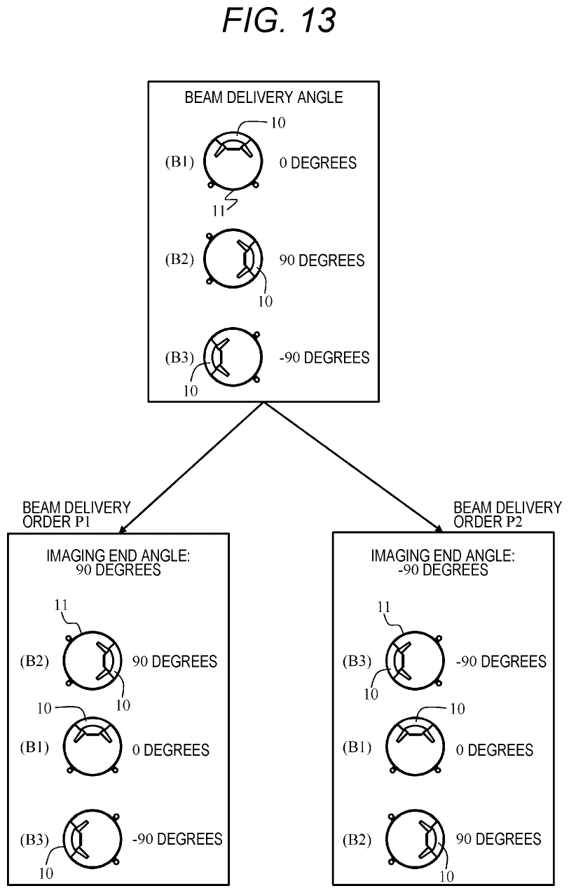

A second embodiment will be described with reference to FIGS. 13 to 15. In the following embodiments including the present embodiment, differences from the first embodiment will be mainly described. In the present embodiment, in the case of extracting a therapeutic beam from a plurality of beam delivery angles, selection of an appropriate beam delivery order and selection of an appropriate imaging parameter are performed.

As illustrated on the upper side of FIG. 13, a case where a particle beam (therapeutic beam) is delivered to a target 18 from a total of three angles as indicated by beam delivery B1 from 0 degrees, beam delivery B2 from 90 degrees, and beam delivery B3 from 270 degrees (−90 degrees) will be described as an example.

In this example, as illustrated on the lower side of FIG. 13, the number of beam delivery orders is two, that is, a beam delivery order P1 and a beam delivery order P2. At first glance, it seems that it is possible to extract the particle beam from the angle of 0 degrees, then extract the particle beam from the angle of 90 degrees, and further extract the particle beam from the angle of 270 degrees (−90 degrees) (the same applies to a case where the angle of 0 degrees→the angle of 270 degrees (−90 degrees)→the angle of 90 degrees), but in this case, the movement amount of the gantry 11 becomes large. If the gantry 11 is moved in either the beam delivery order P1 in which the gantry is moved counterclockwise from the imaging end angle or the beam delivery order P2 in which the gantry 11 is moved clockwise from the imaging end angle, particle beam delivery can be efficiently performed.

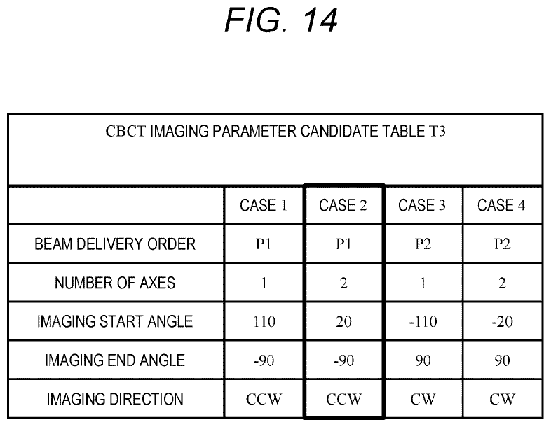

FIG. 14 illustrates an example of a table T3 for managing candidates of the imaging parameter for each case in the example of FIG. 13. Here, a case where the current gantry angle is 30 degrees and the FOV is set to “Small” will be considered.

When the particle beam is delivered from the three beam delivery angles B1 to B3, an appropriate beam delivery order in which the movement amount of the gantry 11 is small is either the beam delivery order P1 or the beam delivery order P2 as described in FIG. 13. In each of the beam delivery orders P1 and P2, there may be a case where the CBCT imaging is performed along one axis and a case where the CBCT imaging is performed along two axes. Therefore, as illustrated in FIG. 14, a total of four candidates of the imaging parameter in Cases 1 to 4 are generated. In the case of the beam delivery order P1, the CBCT imaging along one axis is Case 1, and the CBCT imaging along two axes is Case 2. In the case of the beam delivery order P2, the CBCT imaging along one axis is Case 3, and the CBCT imaging along two axes is Case 4.

In the case of the beam delivery order P1 (in Cases 1 and 2), an angle at which the particle beam is first delivered is 270 degrees (−90 degrees). Therefore, the imaging start angle for the CBCT imaging along one axis of Case 1 is 110 degrees (an angle of 270 degrees (−90 degrees) when rotating counterclockwise by 200 degrees from 110 degrees). Similarly, the imaging start angle for the CBCT imaging along two axes of Case 2 is 20 degrees (imaging in the counterclockwise direction).

In the case of the beam delivery order P2 (in Cases 3 and 4), an angle at which the particle beam is first delivered is 90 degrees. Therefore, the imaging start angle for the CBCT imaging along one axis of Case 3 is 250 degrees (−110 degrees) (an angle of 90 degrees when rotating clockwise by 200 degrees from 250 degrees (−110 degrees)). Similarly, the imaging start angle for the CBCT imaging along two axes of Case 4 is 340 degrees (−20 degrees) (imaging in the clockwise direction).

The CBCT imaging along two axes is usually selected to shorten the treatment time. Therefore, the imaging parameter of any one of Cases 2 and 4 is selected. Since the imaging start angle of Case 2 is 20 degrees that is closest to the current gantry angle of 30 degrees, the imaging parameter of Case 2 is selected. In a case where the CBCT imaging along one axis is performed, the imaging parameter of Case 1 that is the imaging start angle closest to the current gantry angle of 30 degrees is selected.

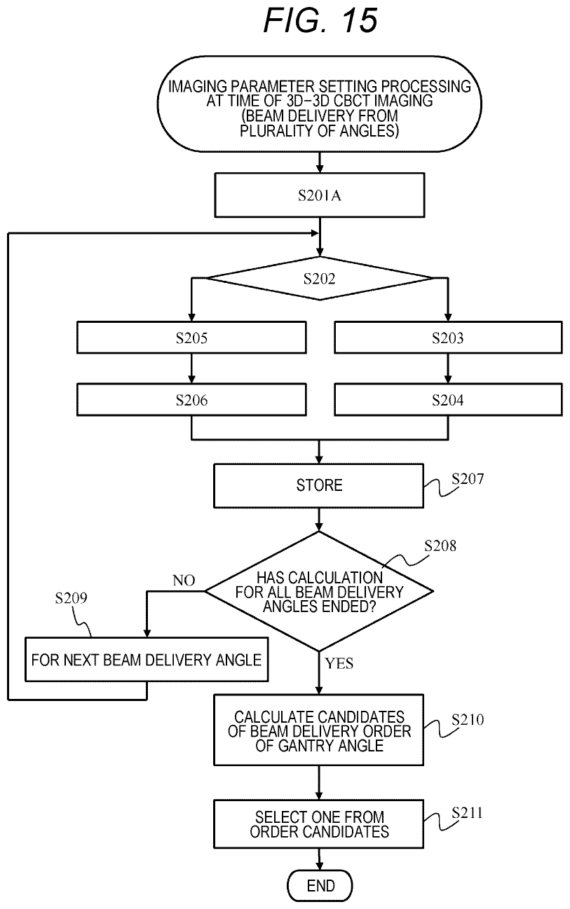

FIG. 15 is a flowchart of processing of calculating the imaging parameter for each beam delivery angle and setting the beam delivery order. Steps S202 to S206 are similar to steps S202 to S206 described in FIG. 10. In step S201A, the control system 50 acquires a plurality of beam delivery angles from the radiotherapy information system 60.

The control system 50 calculates and stores the candidates of the imaging parameter for each beam delivery angle (S207). The control system 50 determines whether or not the calculation of the imaging parameter has ended for all the beam delivery angles (S208). In a case where there is a beam delivery angle for which the imaging parameter has not been calculated (S208: NO), the control system 50 selects one beam delivery angle to be the next calculation target (S209), and returns to step S202.

After calculating the imaging parameter for all the beam delivery angles acquired in step S201A (S208: YES), the control system 50 calculates candidates of the beam delivery order of the gantry angles (S210). The control system 50 selects one beam delivery order in which the movement amount of the gantry 11 is the smallest among the candidates of the beam delivery order (S211).

The present embodiment configured as described above also has the same effects as the first embodiment. Furthermore, in the present embodiment, even in a case where the therapeutic beam is extracted from a plurality of beam delivery angles, the beam delivery order and the imaging parameter can be set such that the movement amount of the gantry 11 is further reduced. Therefore, a consideration time of the user can be shortened to improve usability, and the treatment time can be shortened to reduce a burden on the patient. Furthermore, wear and the like caused by the movement of the gantry 11 can be further reduced, the maintenance frequency can be reduced, and the power consumption can be reduced. Therefore, the operation cost of the radiotherapy system 1000 can be reduced.

### Third Embodiment

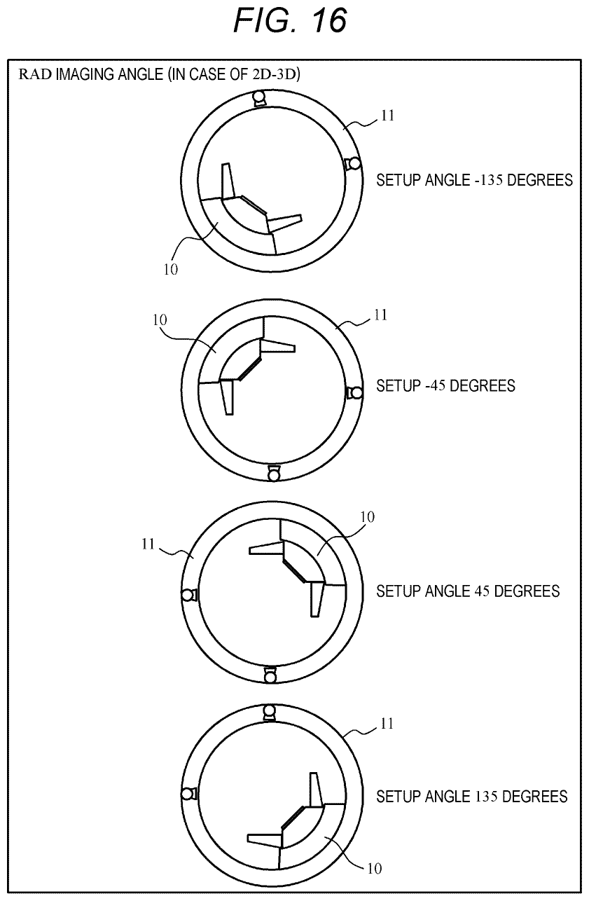

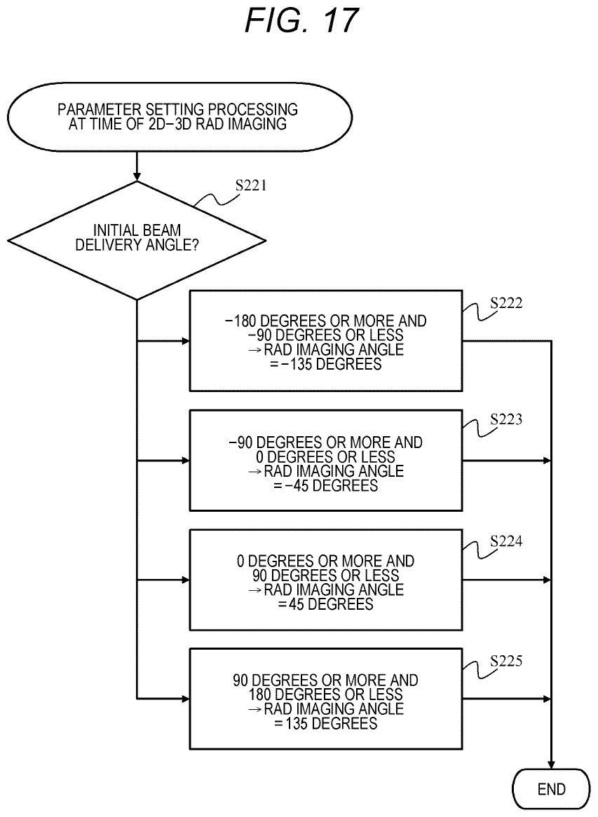

A third embodiment will be described with reference to FIGS. 16 and 17. In the present embodiment, a case where a two-dimensional image of a periphery including a target 18 is acquired using an X-ray imaging device will be described. Imaging for acquiring a two-dimensional image like general X-ray imaging is called RAD imaging.

A 2D-3D method is a method of correcting misalignment between a treatment plan and immediately before the treatment by collating a three-dimensional image obtained by CBCT imaging at the time of creating the treatment plan with a two-dimensional image obtained by RAD imaging immediately before the treatment.

In the RAD imaging, a body tissue including the target 18 is imaged once by the X-ray imaging device to obtain the two-dimensional image.

FIG. 16 illustrates an example of an angle (also referred to as a setup angle) of the RAD imaging. In the RAD imaging of the 2D-3D method, four setup angles of 225 degrees (−135 degrees), 315 degrees (−45 degrees), 45 degrees, and 135 degrees are used to image a patient generally using a combination of front and side views. Therefore, in a section where a beam delivery angle is 180 degrees (−180 degrees) to 270 degrees (−90 degrees), the setup angle of 225 degrees (−135 degrees) is used. In a section where the beam delivery angle is 270 degrees (−90 degrees) to 0 degrees (360 degrees), the setup angle of 315 degrees (−45 degrees) is used. In a section where the beam delivery angle is from 0 degrees to 90 degrees, the setup angle of 45 degrees is used. In a section where the beam delivery angle is 90 degrees to 180 degrees, the setup angle of 135 degrees is used. As described above, the imaging angle (setup angle) is determined by the section in which the beam delivery angle is set.

FIG. 16 illustrates a case where the imaging angle is determined depending on which of the four predetermined sections includes the beam delivery angle. The present invention is not limited thereto, and the RAD imaging may be enabled at an arbitrary angle. For example, imaging may be performed at the same angle as the beam delivery angle determined in the treatment plan. The user may be allowed to select whether to perform imaging at an arbitrary angle or to perform imaging at an angle determined for each section where the beam delivery angle exists.

FIG. 17 is a flowchart illustrating processing of determining the angle (setup angle) of the RAD imaging according to the beam delivery angle.

A control system 50 detects the initial beam delivery angle based on the treatment plan acquired from a radiotherapy information system 60 (S221). In a case where the beam delivery angle is 180 degrees (−180 degrees) or more and less than 270 degrees (−90 degrees), the control system 50 selects the setup angle of 225 degrees (−135 degrees) as the RAD imaging angle (S222). In a case where the beam delivery angle is 270 degrees (−90 degrees) or more and less than 360 degrees (0 degrees), the control system 50 selects the setup angle of 315 degrees (−45 degrees) (S223). In a case where the beam delivery angle is 0 degrees or more and less than 90 degrees, the control system 50 selects the setup angle of 45 degrees (S224). In a case where the beam delivery angle is 90 degrees or more and less than 180 degrees, the control system 50 selects the setup angle of 135 degrees (S225).

The present embodiment configured as described above also has the same effects as the first embodiment. In the present embodiment, in the case of the RAD imaging, the imaging angle of a gantry 11 can be promptly determined and provided to the user, and usability for the user can be improved. Furthermore, in the present embodiment, a movement amount of the gantry 11 from an imaging field to a treatment field can be reduced, a time required for the treatment can be shortened, and a burden on the patient can be reduced.

### Fourth Embodiment

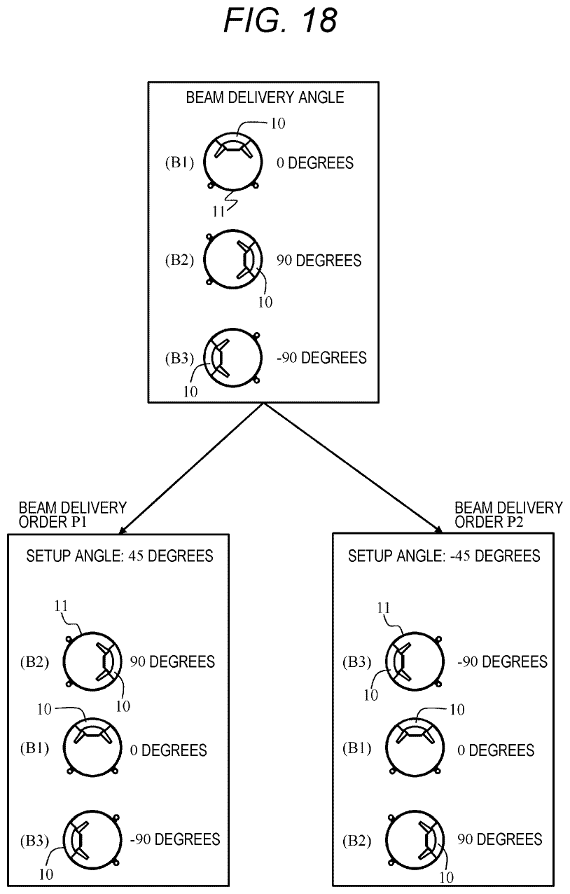

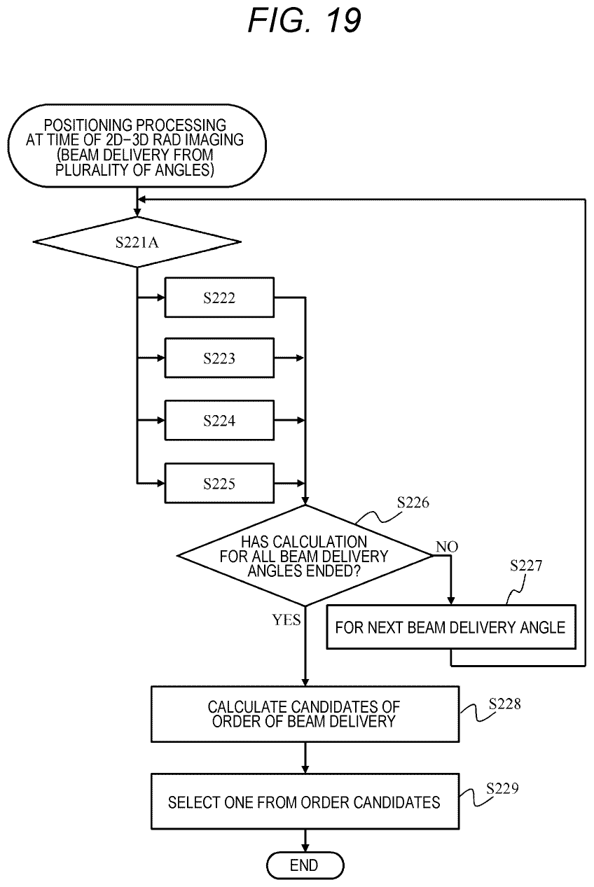

A fourth embodiment will be described with reference to FIGS. 18 and 19. In the present embodiment, in the case of extracting a therapeutic beam from a plurality of beam delivery angles, selection of an appropriate beam delivery order and selection of an appropriate imaging parameter are performed.

As illustrated on the upper side of FIG. 18, a case where a particle beam (therapeutic beam) is delivered to a target 18 from a total of three angles as indicated by beam delivery B1 from 0 degrees, beam delivery B2 from 90 degrees, and beam delivery B3 from 270 degrees (−90 degrees) will be described as an example. The current gantry angle is 30 degrees.

In this example, as illustrated on the lower side of FIG. 18, the number of beam delivery orders is two, that is, a beam delivery order P1 and a beam delivery order P2. At first glance, it seems that it is possible to extract the particle beam from the angle of 0 degrees, then extract the particle beam from the angle of 90 degrees, and further extract the particle beam from the angle of 270 degrees (−90 degrees) (the same applies to a case where the angle of 0 degrees→the angle of 270 degrees (−90 degrees)→the angle of 90 degrees), but in this case, the movement amount of a gantry 11 becomes large. Therefore, if the gantry 11 is moved either in order in a counterclockwise direction (beam delivery order P1) or in order in a clockwise direction (beam delivery order P2), particle beam delivery can be efficiently performed.

In the case of the beam delivery order P1, since the initial beam delivery angle is 90 degrees, a setup angle is set to 45 degrees. In the case of the beam delivery order P2, since the initial beam delivery angle is 270 degrees (−90 degrees), the setup angle is set to 315 degrees (−45 degrees).

FIG. 19 is a flowchart of processing of setting a parameter for RAD imaging. Steps S222 to S225 are similar to steps S222 to S225 described in FIG. 17. In step S221A, a control system 50 learns that the therapeutic beam is extracted from a plurality of beam delivery angles based on a treatment plan acquired from a radiotherapy information system 60.

The control system 50 determines whether or not calculation of the setup angle has ended for all the beam delivery angles (S226). In a case where there is a beam delivery angle for which the setup angle has not been calculated (S226: NO), the control system 50 selects one beam delivery angle to be the next calculation target (S227), and returns to step S221A.

In a case where the setup angle has been calculated for all the beam delivery angles (S226: YES), the control system 50 calculates candidates of the order of the particle beam delivery (S228), and selects one from the order candidates such that the total movement amount of the gantry 11 becomes the shortest (S229).

The present embodiment configured as described above also has the same effects as the first embodiment. In the present embodiment, in a case where the therapeutic beam is extracted from a plurality of beam delivery angles, an appropriate parameter for the RAD imaging can be automatically selected.

### Fifth Embodiment

In the first to fourth embodiments, a case where rotation beyond one full rotation cannot be performed due to a mechanical restriction of a movable range of a gantry 11 has been described. The present embodiment is applied to a case where there is no restriction on the rotation of the gantry 11. That is, the present embodiment can be applied even when the gantry 11 is configured to be rotatable by 360 degrees or more.

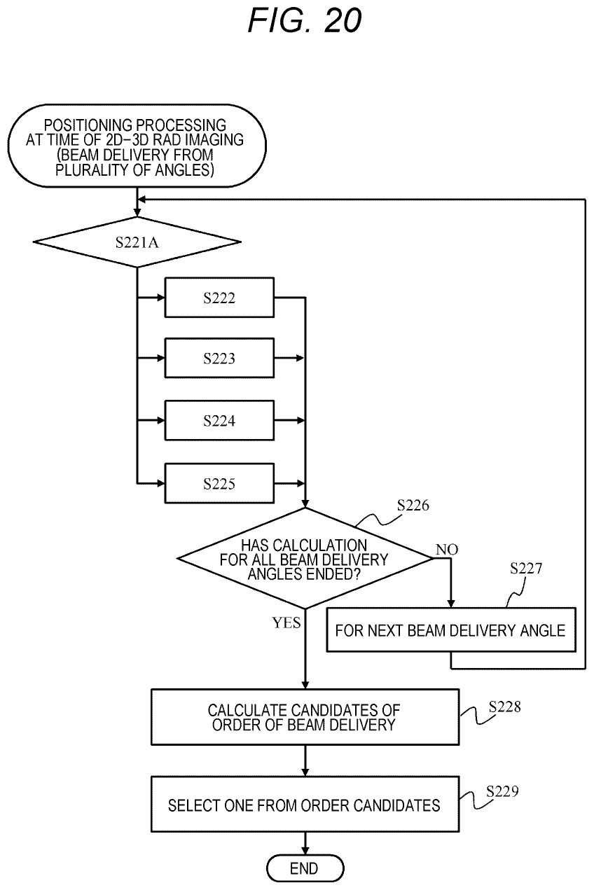

As illustrated in the explanatory view of FIG. 20, in a case where the gantry 11 is configured to be rotatable by 360 degrees or more, the gantry 11 can rotate across a rotation restriction angle TP described in the first to fourth embodiments. In a radiotherapy system in this case, the gantry 11 can rotate by 360 degrees or more clockwise and/or 360 degrees or more counterclockwise.

Examples of the radiotherapy system in which the gantry 11 is rotatable by 360 degrees or more include a particle therapy apparatus in which a circular accelerator is mounted on the gantry 11 and an X-ray therapy apparatus rotatable by 360 degrees or more. In the case of such a radiotherapy system, a parameter selection unit does not need to newly generate candidates for an imaging parameter in which an imaging start angle is the rotation restriction angle. Furthermore, the parameter selection unit can select, as a predetermined imaging parameter, a candidate that reduces the total movement amount of the gantry 11 required for imaging by an imaging device and therapeutic beam delivery by a beam delivery device among the generated candidates of the imaging parameter.

Note that the present invention is not limited to the above-described embodiments. Those skilled in the art can make various additions, modifications, and the like without departing from the scope of the present invention. The above-described embodiments are not limited to the configuration example illustrated in the accompanying drawings. The configurations and the processing methods of the embodiments can be appropriately changed within the scope of achieving the object of the present invention.

For example, it is obvious that the present disclosure includes the following inventions.

### “Expression 1.

A radiotherapy system including a gantry that includes a beam delivery device and an imaging device, the radiotherapy system including:
a parameter selection unit that selects an imaging parameter when the imaging device captures a two-dimensional image including a beam delivery target; and
a control unit that controls the imaging device by using a predetermined imaging parameter selected by the parameter selection unit, in which
the parameter selection unit selects, as the predetermined imaging parameter, an imaging parameter that reduces a movement amount of the gantry to a beam delivery start angle, which is a gantry angle at which therapeutic beam delivery is performed by the beam delivery device, among a plurality of imaging angles prepared in advance.”

### “Expression 2.

A radiotherapy system including a gantry that includes a beam delivery device and an imaging device, the radiotherapy system including:
a parameter selection unit that selects an imaging parameter when the imaging device captures a two-dimensional image including a beam delivery target; and
a control unit that controls the imaging device by using a predetermined imaging parameter selected by the parameter selection unit, in which
the parameter selection unit selects, as the predetermined imaging parameter, an imaging parameter that minimizes a movement amount of the gantry between a beam delivery start angle, which is a gantry angle at which therapeutic beam delivery is performed by the beam delivery device, and a current gantry angle.”

### Reference Signs List

1 therapeutic beam generator
2 linac
3 synchrotron
4, 6, 8 bending magnet
5 particle beam transport system
7 particle beam path
9 treatment room
12, 13 X-ray generator
14, 15 X-ray detector
16 beam delivery target
17 couch
18 target
50 control system
51 central control device
52 accelerator control device
53 gantry control device
54 beam delivery control device
55 X-ray imaging control device
56 couch control device
57 imaging control device
58 image generation device
59 treatment information control device
60 radiotherapy information system
1000 radiotherapy system

1. A radiotherapy system including a gantry that includes a beam delivery device and an imaging device, the radiotherapy system comprising:
a parameter selection unit that selects an imaging parameter including an imaging start angle which is a gantry angle when the imaging device starts imaging, an imaging end angle which is a gantry angle when the imaging device ends imaging, and a rotation direction of the gantry; and
a control unit that controls the imaging device by using a predetermined imaging parameter selected by the parameter selection unit, wherein the parameter selection unit generates one or more candidates of the imaging parameter and selects, as the predetermined imaging parameter, a candidate that reduces a total movement amount of the gantry required for imaging by the imaging device and therapeutic beam delivery by the beam delivery device among the generated candidates of the imaging parameter.

2. The radiotherapy system according to claim 1, wherein the parameter selection unit generates one or more candidates of the imaging parameter in which a difference between a beam delivery start angle and the imaging end angle is within a predetermined range, the beam delivery start angle being a gantry angle at which the therapeutic beam delivery is performed by the beam delivery device, and selects, as the predetermined imaging parameter, a candidate that reduces the total movement amount of the gantry required for imaging by the imaging device and the therapeutic beam delivery by the beam delivery device among the generated candidates of the imaging parameter.

3. The radiotherapy system according to claim 2, wherein the parameter selection unit selects an imaging parameter that reduces a total movement amount from a current angle of the gantry to the beam delivery start angle among the generated candidates of the imaging parameter.

4. The radiotherapy system according to claim 2, wherein the parameter selection unit selects, as the predetermined imaging parameter, a candidate of the imaging parameter that reduces a total movement amount of the gantry from a current angle of the gantry until transitioning to the therapeutic beam delivery by the beam delivery device through the imaging by the imaging device, among the generated candidates of the imaging parameter.

5. The radiotherapy system according to claim 1, wherein a rotation restriction angle that restricts rotation beyond one full rotation is set for the gantry, and the parameter selection unit generates at least one candidate of the imaging parameter in which the rotation restriction angle is not included in a range from the imaging start angle to the imaging end angle.

6. The radiotherapy system according to claim 1, wherein a rotation restriction angle that restricts rotation beyond one full rotation is set for the gantry, and the parameter selection unit generates, in a case where the rotation restriction angle is within a range from the imaging start angle to the imaging end angle among the generated imaging parameters, other candidates of the imaging parameter in which the rotation restriction angle is not included in the range from the imaging start angle to the imaging end angle, and selects, as the predetermined imaging parameter, a candidate that reduces a total movement amount of the gantry required for the imaging by the imaging device and the therapeutic beam delivery by the beam delivery device among the generated other candidates of the imaging parameter.

7. The radiotherapy system according to claim 6, wherein the parameter selection unit generates the other candidates of the imaging parameter such that the imaging start angle is the rotation restriction angle.

8. The radiotherapy system according to claim 1, wherein the imaging device includes a plurality of X-ray generation units provided so as to sandwich a beam delivery unit, and a plurality of X-ray imaging units provided in the gantry at positions facing the X-ray generation units, and imaging is performed by at least one set of the X-ray generation unit and the X-ray imaging unit among a plurality of sets of the X-ray generation units and the X-ray imaging units disposed to face each other

9. The radiotherapy system according to claim 8, wherein the parameter selection unit generates a candidate of the imaging parameter in a case where imaging is performed by any one set of the X-ray generation unit and the X-ray imaging unit among the plurality of sets of the X-ray generation units and the X-ray imaging units disposed to face each other, and a candidate of the imaging parameter in a case where imaging is performed by all the sets of the X-ray generation units and the X-ray imaging units among the plurality of sets of the X-ray generation units and the X-ray imaging units disposed to face each other.

10. The radiotherapy system according to claim 1, wherein the imaging device is configured to select any one of a two-dimensional image acquisition mode in which a two-dimensional image including a beam delivery target is acquired by an X-ray and a three-dimensional image acquisition mode in which a three-dimensional image including the beam delivery target is acquired by an X-ray.

11. The radiotherapy system according to claim 10, wherein the parameter selection unit generates, in a case where the two-dimensional image acquisition mode is selected, a candidate of the imaging parameter that reduces a movement amount of the gantry to a beam delivery start angle among a plurality of imaging angles prepared in advance, the beam delivery start angle being a gantry angle at which the therapeutic beam delivery is performed by the beam delivery device.

12. The radiotherapy system according to claim 2, wherein a case where the difference between the beam delivery start angle, which is a gantry angle at which the therapeutic beam delivery is performed by the beam delivery device, and the imaging end angle is within the predetermined range includes a case where the beam delivery start angle and the imaging end angle match each other.

13. The radiotherapy system according to claim 1, wherein the control unit causes the beam delivery device to deliver the therapeutic beam to a beam delivery target based on a misalignment amount between a current image of the beam delivery target captured by the imaging device and a reference image at the time of treatment planning.

14. A method for controlling a radiotherapy system including a gantry that includes a beam delivery device and an imaging device, the method comprising:
generating candidates of an imaging parameter including an imaging start angle which is a gantry angle when the imaging device starts imaging, an imaging end angle which is a gantry angle when the imaging device ends imaging, and a rotation direction of the gantry; and
selecting, as a predetermined imaging parameter, a candidate that reduces a total movement amount of the gantry required for imaging by the imaging device and therapeutic beam delivery by the beam delivery device among the generated candidates of the imaging parameter.
